(19) United States
(12) Patent Application Publication
(10) Pub. No.: US 2023/0363748 A1
(43) Pub. Date: Nov. 16, 2023

(54) BATTERY DISCHARGE MECHANISM FOR SURGICAL INSTRUMENTS

(71) Applicant: Covidien LP, Mansfield, MA (US)

(72) Inventor: Xingrui Chen, Glastonbury, CT (US)

(21) Appl. No.: 18/314,465

(22) Filed: May 9, 2023

Related U.S. Application Data
(60) Provisional application No. 63/341,005, filed on May 12, 2022.

(51) Int. Cl. A61B 17/00; H02J 7/00; H01M 50/247; H01M 10/42

(52) U.S. Cl. CPC A61B 17/00; H02J 7/0063; H01M 50/247; H01M 10/425; A61B 2017/00734; H01M 2220/30

(57) ABSTRACT

A surgical instrument includes a handle assembly and a battery assembly configured to removably couple to the handle assembly. The handle assembly includes a handle housing, an electronic component within the housing, a handle finger extending from the handle housing, and an electrical contact electrically coupled to the electronic component. The battery assembly includes a battery housing, a battery disposed in the battery housing, a battery contact electrically coupled to the battery, and a discharge circuit board. The discharge circuit board is configured to discharge the battery and is movable within the battery housing from a first position to a second position by the handle finger upon coupling the battery assembly to the handle assembly.

### CROSS-REFERENCE TO RELATED APPLICATIONS

This application claims the benefit of, and priority to, U.S. Provisional Patent Application Ser. No. 63/341,005, filed on May 12, 2022, the entire contents of which are incorporated by reference herein.

### BACKGROUND

Powered surgical instruments utilize one or more electrical components, such as circuit boards, sensors, motors, etc. to operate various functions of the instrument. Handheld powered surgical instruments may have a corded power supply configuration or may be battery operated. Some battery-operated surgical instruments are powered by a removable battery pack, which is typically discarded after use. Many governing bodies across the globe require batteries to be fully discharged by the time they reach the end of their waste stream. Accordingly, a need exists for a low-cost mechanism that ensures the full discharge of a battery subsequent to the battery's use and prior to disposal.

### SUMMARY

The present disclosure provides a surgical instrument that includes a handle assembly and a battery assembly configured to removably couple to the handle assembly. The handle assembly includes a handle housing, an electronic component within the housing, a handle finger extending from the handle housing, and first and second electrical terminals electrically coupled to the electronic component. The battery assembly includes a battery housing, a battery cell disposed in the battery housing, first and second battery contacts electrically coupled to the battery cell, and a discharge circuit board. The discharge circuit board is configured to discharge the battery cell and is movable within the battery housing from a first position to a second position by the handle finger upon coupling the battery assembly to the handle assembly. The discharge circuit board includes a first contact pad electrically coupled to the first battery contact, a second contact pad electrically coupled to the second battery contact, and a switch including a first end extending from the second contact pad and a second end movable relative to the first contact pad between a first condition, where the second end is in conductive contact with the first contact pad, and a second condition, where the second end is not in conductive contact with the first contact pad.

In an aspect, the surgical instrument may include an elongated shaft extending from the handle assembly and an end effector operably coupled to the elongated shaft.

In an aspect, the battery assembly may include a retainer configured to maintain the discharge circuit board in the second position after the battery assembly is removed from the handle assembly. Additionally, or alternatively, the retainer may be configured to maintain the discharge circuit board in the first position prior to the battery assembly being coupled to the handle assembly.

In an aspect, the discharge circuit board may further include a plunger positioned under the switch and movable through an opening defined through the discharge circuit board. The plunger may be configured to move the switch upward to disengage the switch from contact with the first contact pad. Additionally or alternatively, the handle finger includes a ramped surface and the plunger may be configured to slide along the ramped surface as the battery assembly is being connected to the handle assembly.

In an aspect, the first contact pad may be electrically coupled to the first battery contact and the second contact pad may be electrically coupled to the second battery contact when the discharge circuit board is in the first position and the second position.

In an aspect, the discharge circuit board may be configured to longitudinally slide along a track of the battery assembly.

In an aspect, the battery housing may include a battery connector and the handle housing may include a handle connector configured to releasably mate with the battery connector.

In an aspect, the first battery contact may be electrically coupled to a first terminal of the battery cell and the second battery contact may be electrically coupled to a second terminal of the battery cell.

Another aspect of the present disclosure provides a battery assembly for use with a surgical instrument. The battery assembly includes a housing, a battery cell disposed within the battery housing, first and second battery contacts electrically coupled to the battery cell, and a discharge circuit board. The discharge circuit board is movable within the battery housing between a first position in the battery housing and a second position in the battery housing. The discharge circuit board includes a first contact pad electrically coupled to the first battery contact, a second contact pad electrically coupled to the second battery contact, and a switch. The switch includes a first end extending from the second contact pad and a second end movable relative to the first contact pad between a first condition, where the second end is in conductive contact with the first contact pad, and a second condition, where the second end is not in conductive contact with the first contact pad. The discharge circuit board is configured to discharge the battery cell when the switch is in the second condition and the discharge circuit board is in the second position.

In an aspect, the battery assembly may further include a retainer configured to maintain the discharge circuit board in the first position and the second position.

In an aspect, the battery assembly may further include a track defined in the battery housing, and the discharge circuit board may be configured to longitudinally slide along the track.

In an aspect, the first battery contact may be electrically coupled to a first terminal of the battery cell and the second battery contact may be electrically coupled to a second terminal of the battery cell.

In an aspect, the discharge circuit board may further include a plunger positioned under the switch and movable through an opening defined through the discharge circuit board. The plunger may be configured to move the switch upward to disengage the switch from contact with the first contact pad.

In an aspect, the first contact pad may be electrically coupled to the first battery contact and the second contact pad may be electrically coupled to the second battery contact when the discharge circuit board is in the first position and the second position.

Another aspect of the present disclosure provides a surgical instrument that includes a handle assembly and a battery assembly configured to removably couple to the handle assembly. The battery assembly includes a battery housing, a battery cell disposed within the battery housing, first and second battery contacts electrically coupled to the battery cell, and a discharge circuit board. The discharge circuit board is movable within the battery housing between a first position in the battery housing and a second position in the battery housing. The discharge circuit board includes a first contact pad electrically coupled to the first battery contact, a second contact pad electrically coupled to the second battery contact, and a switch. The switch includes a first end extending from the second contact pad and a second end movable relative to the first contact pad between a first condition, where the second end is in conductive contact with the first contact pad, and a second condition, where the second end is not in conductive contact with the first contact pad. The discharge circuit board is configured to discharge the battery cell when the switch is in the second condition and the discharge circuit board is in the second position.

In an aspect, the battery assembly may include a retainer configured to maintain the discharge circuit board in the first position prior to the battery assembly being coupled to the handle assembly and maintain the discharge circuit board in the second position after the battery assembly is removed from the handle assembly.

### BRIEF DESCRIPTION OF THE DRAWINGS

Various aspects of the disclosed surgical instrument are described herein below with reference to the drawings, wherein:

FIG. 1 is a perspective view of a powered surgical instrument including a handle assembly and a battery assembly according to an embodiment of the present disclosure;

FIG. 2 is a side perspective view of a first cross-section of the handle assembly and the battery assembly of the powered surgical instrument of FIG. 1 in a pre-installation state;

FIG. 3 is a side perspective view of a second cross-section of the handle assembly and the battery assembly of the powered surgical instrument of FIG. 1 in the pre-installation state;

FIG. 4 is a side perspective view of a third cross-section of the handle assembly and the battery assembly of the powered surgical instrument of FIG. 1 in a post-installation state;

FIG. 5 is a side perspective view of the third cross-section of the handle assembly and the battery assembly of the powered surgical instrument of FIG. 1 in a disposal state;

FIG. 6 is a side perspective view of a fourth cross-section of the handle assembly and the battery assembly of the powered surgical instrument of FIG. 1 in the disposal state;

FIG. 7 is a rear perspective view of the handle assembly and the battery assembly of the powered surgical instrument of FIG. 1, with parts removed, in the disposal state;

FIG. 8 is a perspective view of a powered surgical instrument including a handle assembly and a battery assembly in a pre-installation state according to another embodiment of the present disclosure;

FIG. 9 is a perspective, cross-sectional, view of a portion of the handle assembly of the powered surgical instrument of FIG. 8;

FIG. 10 is a parts separated view of the battery assembly of the powered surgical instrument of FIG. 8 in the pre-installation state;

FIG. 11 is a side cross-sectional view of the battery assembly of the powered surgical instrument of FIG. 8 in the pre-installation state illustrating a discharge circuit board in a first position;

FIG. 12 is a rear perspective view of the battery assembly of the powered surgical instrument of FIG. 8 with parts removed and in the pre-installation state illustrating the discharge circuit board in the first position;

FIG. 13 is a side perspective view of components of the battery assembly of the powered surgical instrument of FIG. 8;

FIG. 14 is a side, cross-sectional, view, from a first side, of a portion of the battery assembly connected to the handle assembly of the powered surgical instrument of FIG. 8 in a post-installation state with the discharge circuit board in a second position;

FIG. 15 is a cross-sectional view, from a rear side, of a portion of the battery assembly connected to the handle assembly of the powered surgical instrument of FIG. 8 in a post-installation state with portions of the battery assembly removed to illustrate the discharge circuit board in the second position;

FIG. 16 is a side, cross-sectional, view of the battery assembly of the powered surgical instrument of FIG. 8 in a disposal state with the discharge circuit board in the second position;

FIG. 17 is a perspective view of the battery assembly of the powered surgical instrument of FIG. 8 in the disposal state with parts removed to illustrate the discharge circuit board in the second position;

FIG. 18 is a rear perspective view of the battery assembly of the powered surgical instrument of FIG. 8 in the disposal state with parts removed to illustrate the discharge circuit board in the second position;

FIG. 19 is a perspective view of the battery assembly of the powered surgical instrument of FIG. 8 in a pre-installation state, with parts removed, including a discharge circuit board having a detect switch in accordance with another embodiment of the present disclosure;

FIG. 20 is a side, cross-sectional, view, from a first side, of a portion of the battery assembly of FIG. 19 connected to the handle assembly of the powered surgical instrument of FIG. 8 in a post-installation state with the discharge circuit board in a second position; and

FIG. 21 is a side cross-sectional view of the battery assembly including the discharge circuit board of FIG. 19 in a disposal state after removal of the battery assembly from the powered surgical instrument.

### DETAILED DESCRIPTION

The disclosed powered surgical instruments are described in detail with reference to the drawings in which like reference numerals designate identical or corresponding elements in each of the several views. However, it is to be understood that the aspects of the disclosure described herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during usage of the device in a customary manner, while the term “distal” is used generally to refer to that portion of the device that is farther from the clinician during usage of the device in a customary manner. Further, the term “clinician” is used generally to refer to medical personnel including doctors, nurses, and support personnel.

Many governing bodies across the globe require batteries (e.g., lithium cells) to be fully discharged by the time they reach the end of their waste stream. Handheld surgical instruments can utilize removable battery packs to power the instrument during use in a procedure. If the battery pack is removed from the handheld device, thereby disconnecting the electrical load, prior to being fully discharged, the battery will not meet this requirement and can reach the end of its waste stream with electrical energy still available. The presently described surgical instruments include a mechanism for guaranteeing full discharge of the battery irrespective of whether the battery remains connected to the surgical instrument or is removed from the surgical instrument after use.

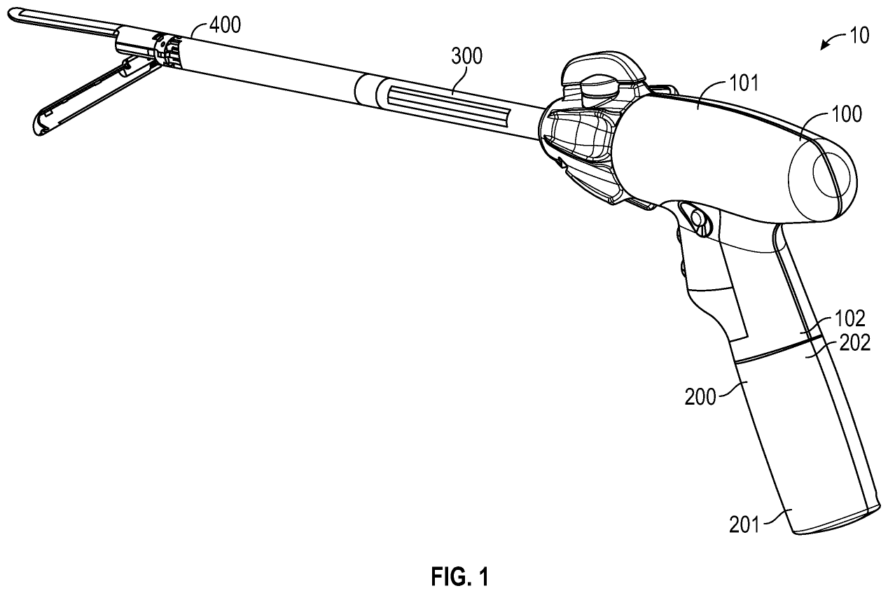

Referring initially to FIG. 1, an electromechanical, hand-held, powered surgical instrument 10, in accordance with aspects of the disclosure includes a handle assembly 100, an elongated shaft 300 extending from the handle assembly 100, and an end effector 400 coupled to a distal end of the elongated shaft 300. The end effector 400 is configured for actuation and manipulation by the handle assembly 100. Surgical instrument 10 may be any battery powered instrument, handheld or robotic, such as a powered surgical stapler, grasper, cutter, electrosurgical device, etc.

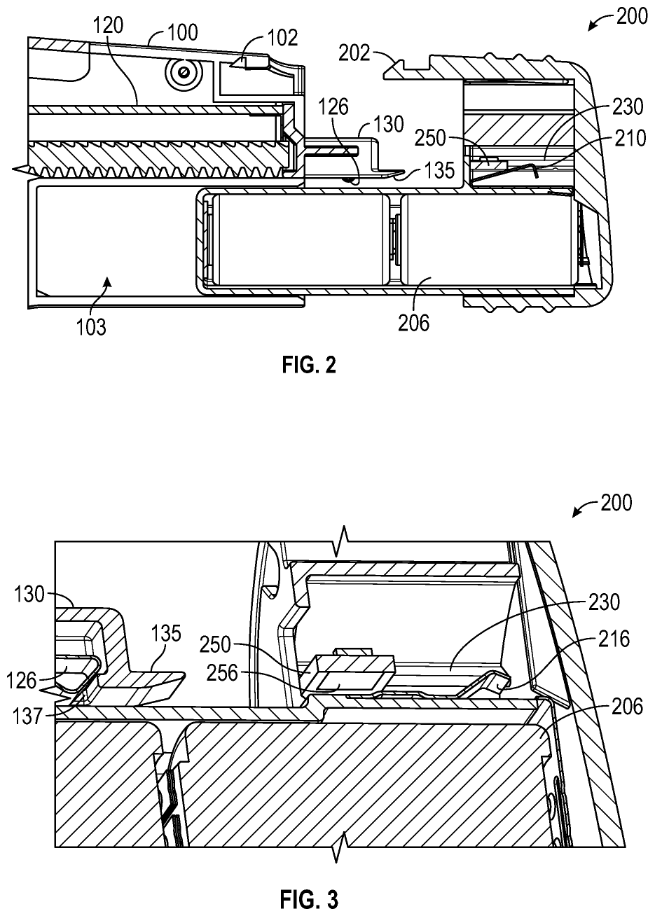

Handle assembly 100 includes a handle housing 101, which houses various electronic component(s) 120 (e.g., circuit boards, motors, sensors, controllers, etc. FIG. 2) and mechanical components configured to control operation of the surgical instrument 10. The surgical instrument 10 additionally includes a battery assembly 200 which is configured to removably couple to the handle assembly 100 and supply power to the electronic components 120 of the surgical instrument 10. In an aspect, the handle housing 101 defines an opening 103 (FIG. 2) configured to receive at least a portion of the battery assembly 200 therein. In aspects, the handle assembly 100 includes a handle connector 102 which is configured to releasably engage a battery connector 202 formed by, or coupled to, a battery housing 201 of the battery assembly 200 to secure the battery assembly 200 to the handle housing 101. The handle connector 102 and battery connector 202 may be any suitable connection mechanism that enables releasable attachment of two components (e.g., press-clip, threading, etc.).

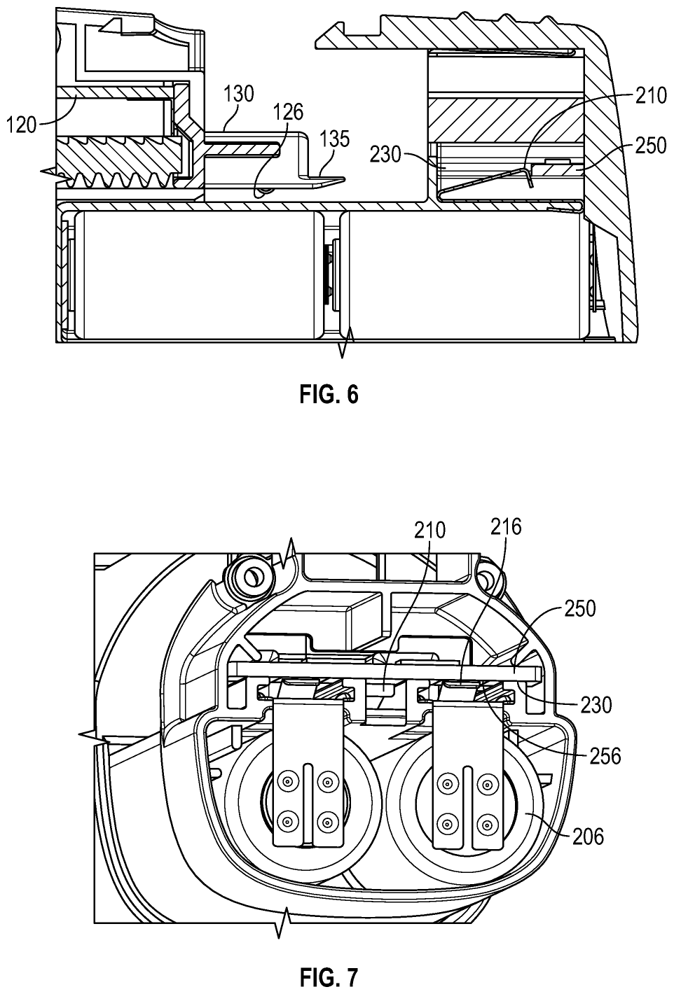

With reference to FIG. 2, the battery assembly 200 includes at least one battery cell 206 disposed within the battery housing 201, and a battery contact 216 conductively coupled to a terminal of the battery cell 206. The battery cell 206 is configured to electrically couple to the electronic component 120 of the surgical instrument 10 when the battery assembly 200 is coupled to the handle assembly 100 to form a circuit therewith and provide power to the electrical components (e.g., the electronic component 120, motors, sensors, etc.) of the surgical instrument 10. Only a single battery contact 216 is illustrated and described for brevity, but it is appreciated that the battery assembly 200 may include a plurality of battery contacts 216 or independent positive and negative terminal contacts (e.g., corresponding to positive and negative terminals of the battery cell 206, respectively). The battery assembly 200 also includes a discharge circuit board 250 which is configured to discharge the battery cell 206 after the battery cell 206 is used with the surgical instrument 10 (e.g., when the battery assembly 200 is in a disposal state, FIGS. 6-8), as described in further detail below.

The discharge circuit board 250 contains a passive resistive load which is conductively coupled to a contact pad 256 (FIGS. 5 and 7) and is movable within the battery housing 201 between a first position (FIGS. 2 and 3) and a second position. The passive resistive load may include one or more resistors. The contact pad 256 may be disposed on an underside of the discharge circuit board 250. In an aspect, the battery housing 201 defines a track 230 (FIGS. 3 and 7) within which the discharge circuit board 250 is configured to longitudinally slide between the first position and the second position. As with the battery contact 216, only a single contact pad 256 is illustrated and described for brevity, but it is appreciated that the discharge circuit board 250 may include two contact pads 256 or independent positive and negative terminal contacts (e.g., corresponding to positive and negative terminals of the battery cell 206, respectively).

The battery assembly 200 further includes a spring clip 210 or any other suitable retaining device that is positioned relative to the discharge circuit board 250 such that the spring clip 210 maintains the position of the discharge circuit board 250 in the first position prior to the battery assembly 200 ever being coupled to any handle assembly 100. The spring clip 210 also maintains the discharge circuit board 250 in the second position after the battery assembly 200 is removed from the handle assembly 100.

The handle assembly 100 includes a handle finger 130 extending from the handle housing 101 and a protrusion 135 extending from the handle finger 130. An electrical contact 126 is electrically coupled to the electronic component 120 and is configured to conductively contact a battery contact 216 of the battery assembly 200 to close the electrical circuit, thereby providing electrical power to the electronic components (e.g., electronic component 120) of the handle assembly 100 when the battery assembly 200 is coupled to the handle assembly 100. In an aspect, the electrical contact 126 extends through an opening 137 (FIG. 7) defined through the handle finger 130. Only a single electrical contact 126 is illustrated and described for brevity, but it is appreciated that the electronic component 120 may include two electrical contacts 126 or independent positive and negative terminal contacts (e.g., corresponding to positive and negative terminals of the battery cell 206, respectively).

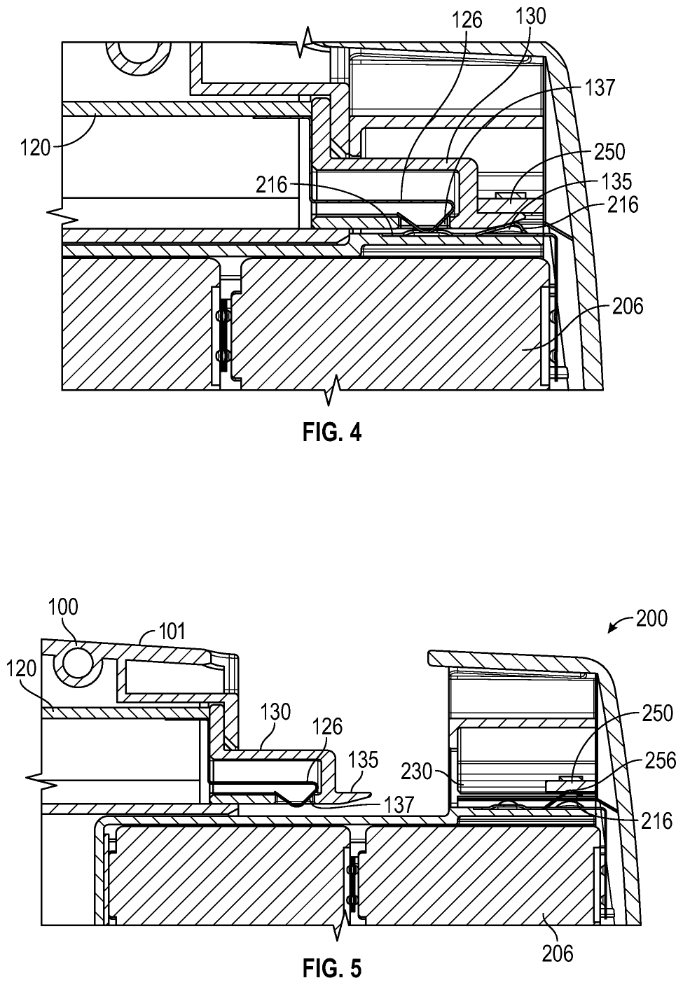

FIG. 3 illustrates the battery assembly 200 in a pre-installation state where, prior to the first use of the battery assembly 200, the discharge circuit board 250 is in the first position, and is maintained in this position by the spring clip 210. In the first position, the contact pad 256 is physically distanced from the battery contact 216, and therefore not in conductive contact therewith. After coupling the battery assembly 200 to the handle assembly 100, the battery assembly 200 is transitioned to a post-installation state (FIG. 4). During coupling of the battery assembly 200 to the handle assembly 100 (e.g., during the transition from the pre-installation state to the post-installation state), the handle finger 130 depresses (or otherwise disengages) the spring clip 210 and urges the discharge circuit board 250 from the first position toward the second position. In addition to urging the discharge circuit board 250 from the first position toward the second position, the protrusion 135 of the handle finger 130 slides under the discharge circuit board 250 to physically cover the contact pad 256 of the discharge circuit board 250, thereby preventing the contact pad 256 from being able to make conductive contact with the battery contact 216. In aspects, the battery contact 216 is moved (e.g., depressed) by the protrusion 135 to physically distance the battery contact 216 from the contact pad 256 in addition to, or in lieu of, the physical covering of the contact pad 256 by the protrusion 135. When the battery assembly 200 is in the post-installation state, the electrical contact 126 is electrically coupled to the battery contact 216, thereby forming the circuit and supplying power to the electronic component 120.

FIGS. 5-7 illustrate the battery assembly in a disposal state, that is, a state in which the battery assembly 200 has been coupled to, and then subsequently removed from, the handle assembly 100. In the disposal state, the discharge circuit board 250 is in the second position and is maintained in the second position by the spring clip 210. In addition, because the protrusion 135 of the handle finger 130 is no longer covering the contact pad 256 of the discharge circuit board 250, and no longer depressing the battery contact 216 to physically separate the battery contact 216 from the contact pad 256, the contact pad 256 is electrically coupled to the battery contact 216. When the contact pad 256 is electrically coupled to the battery contact 216 (e.g., when a positive terminal of the contact pad 256 is contacting a positive terminal of the battery contact 216 and/or a negative terminal of the contact pad 256 is contacting a negative terminal of the battery contact 216), a circuit is formed which enables the discharge circuit board 250 to discharge the battery cell 206. The discharge circuit board 250 will discharge the battery cell 206 until no more electrical energy is left in the battery cell 206 of the battery assembly 200.

The time to fully discharge the battery cell 206 of the battery assembly 200 depends on the capacity of the battery cell 206 and the resistive load of the discharge circuit board 250. In aspects, the resistive load of the discharge circuit board 250 may be selectable, manually or automatically, to control the discharge rate. Resistors forming the resistive load of the discharge circuit board 250 may be implemented in series and parallel to distribute electrical power as well as add safety redundancies to the discharging function of the discharge circuit board 250.

After being in the disposal state, if the battery assembly 200 is recoupled to the handle assembly 100, the protrusion 135 of the handle finger 130 will slide under the discharge circuit board 250 between the contact pad 256 and the battery contact 216 to break the conduction connection therebetween and enable reconnection of the electrical contact 126 to the battery contact 216.

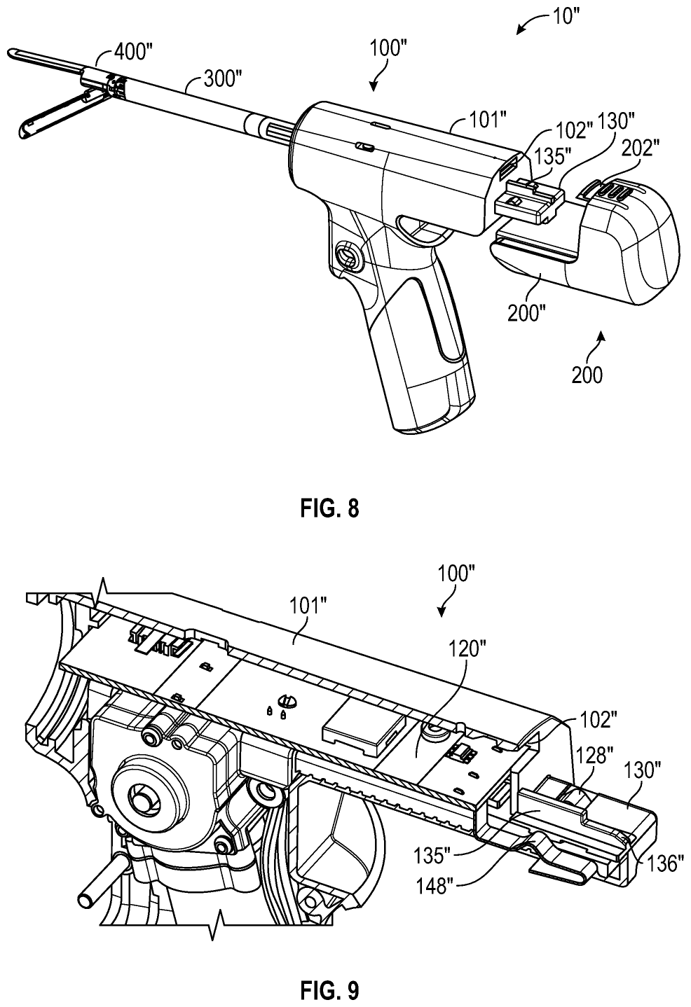

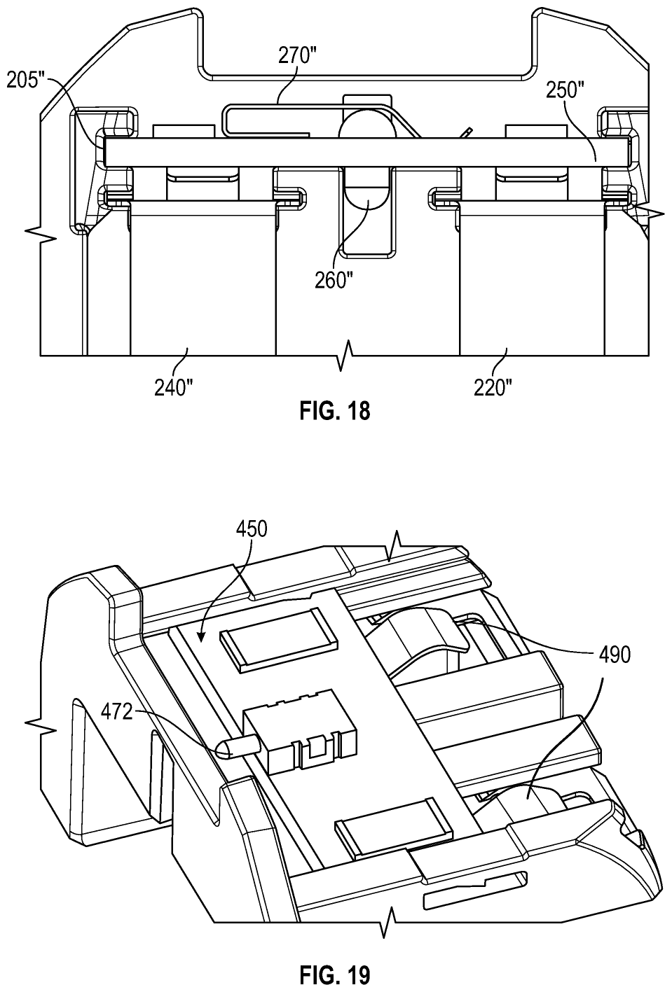

Referring now to FIGS. 8-18, an electromechanical, hand-held, powered surgical instrument 10″, in accordance with aspects of the disclosure includes a handle assembly 100″, an elongated shaft 300″ extending from the handle assembly 100″, and an end effector 400″ coupled to a distal end of the elongated shaft 300″. The end effector 400″ is configured for actuation and manipulation by the handle assembly 100″. Surgical instrument 10″ may be any battery powered instrument, handheld or robotic, such as a powered surgical stapler, grasper, cutter, electrosurgical device, etc.

Handle assembly 100″ includes a handle housing 101″, which houses various electronic component(s) 120″ (e.g., circuit boards, motors, sensors, controllers, etc., FIG. 9) and mechanical components configured to control operation of the surgical instrument 10″. The surgical instrument 10″ additionally includes a battery assembly 200″ which is configured to removably couple to the handle assembly 100″ and supply power to the electronic components 120″ of the surgical instrument 10″. In aspects, the handle assembly 100″ includes a handle connector 102″ which is configured to releasably engage a battery connector 202″ formed by, or coupled to, a battery housing 201″ of the battery assembly 200″ to secure the battery assembly 200″ to the handle housing 101″. The handle connector 102″ and battery connector 202″ may be any suitable connection mechanism that enables releasable attachment of two components (e.g., press-clip, threading, etc.). In aspects, the handle assembly 100″ defines a handle finger 130″ configured to be positioned into a slot 213″ defined by the battery housing 201″ of the battery assembly 200″.

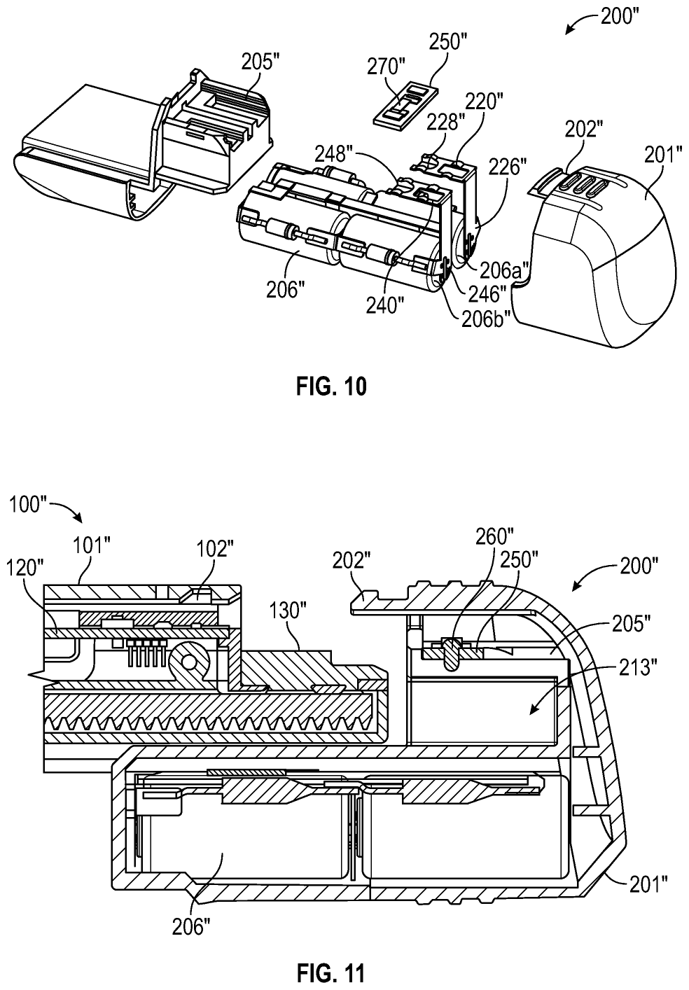

With reference to FIGS. 10, the battery assembly 200″ includes one or more battery cells 206″ disposed within the battery housing 201″. The battery cell 206″ is configured to electrically couple to the electronic component 120″ of the surgical instrument 10″ when the battery assembly 200″ is coupled to the handle assembly 100″ to form a circuit therewith and provide power to the electrical components (e.g., the electronic component 120″, motors, sensors, etc.) of the surgical instrument 10″. The battery assembly 200″ also includes a discharge circuit board 250″ which is configured to discharge the battery cell 206″ after the battery cell 206″ is used with (e.g., connected to), and disconnected from, the surgical instrument 10″ (e.g., when the battery assembly 200″ is in a disposal state), as described in further detail below.

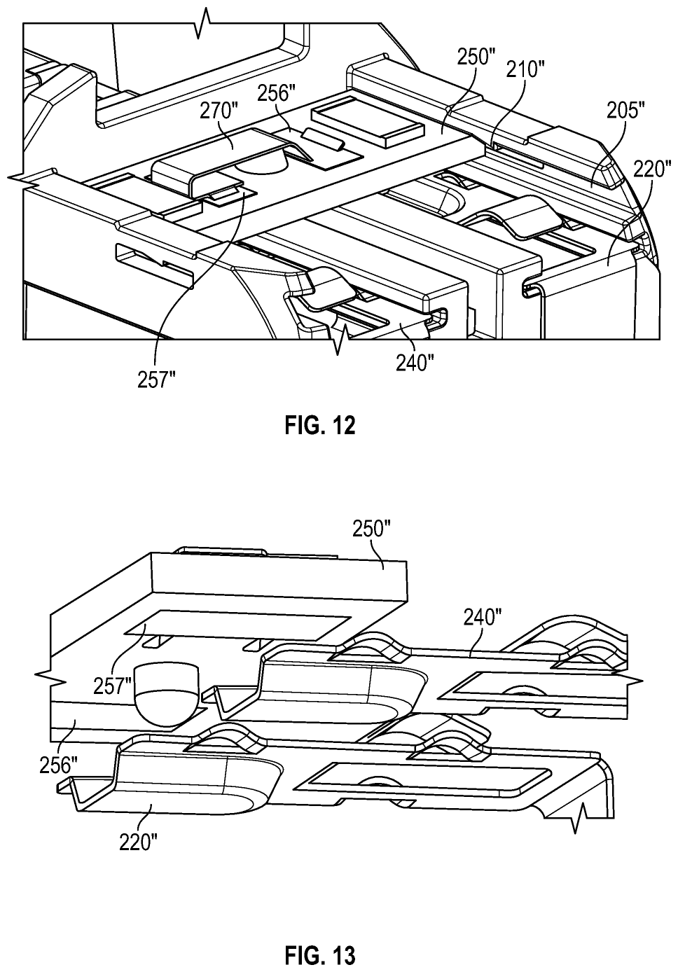

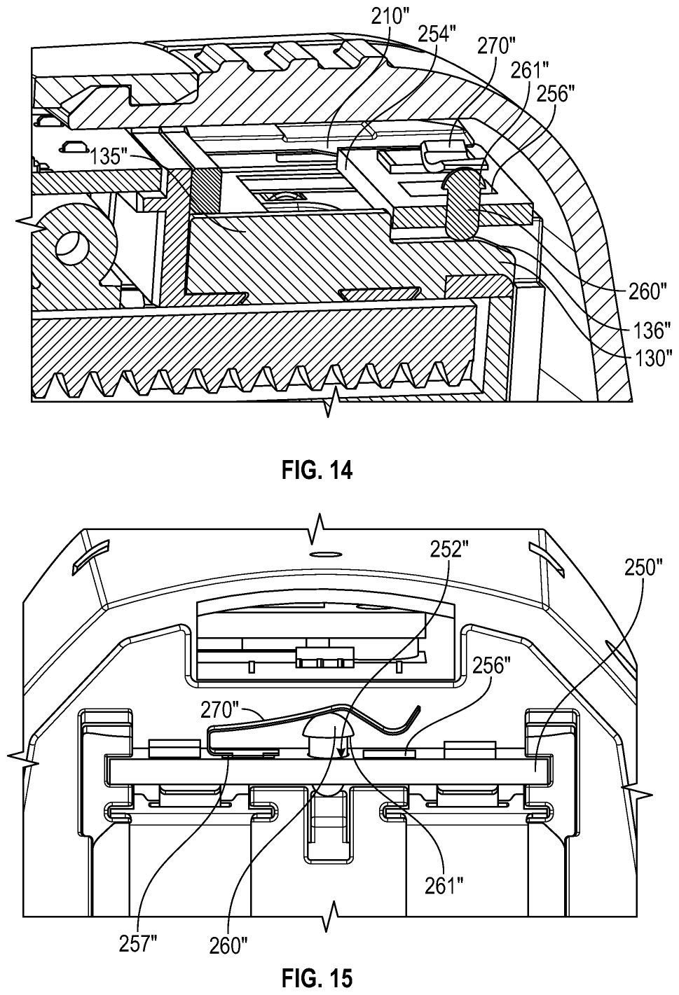

The discharge circuit board 250″ contains a passive resistive load which is conductively coupled to a first contact pad 256″ and a second contact pad 257″ (FIGS. 12 and 13). The discharge circuit board 250″ is movable within the battery housing 201″ between a first position (FIGS. 11-13) and a second position (FIGS. 14-18). In particular, the discharge circuit board 250″ remains in the first position until the battery assembly 200″ is connected to the handle assembly 100″ and is moved to the second position when the battery assembly 200″ is connected to the handle assembly 100″ and remains in the second position after the battery assembly 200″ is removed from the handle assembly 100″. The passive resistive load of the discharge circuit board 250″ may include one or more resistors. In an aspect, a portion of the battery housing 201″ defines a track 205″ within which the discharge circuit board 250″ is configured to longitudinally slide between the first position and the second position.

The discharge circuit board 250″ also includes a plunger 260″ positioned between the first contact pad 256″ and the second contact pad 257″ an extending through an opening 252″ (FIG. 15) defined through the discharge circuit board 250″. The plunger 260″ is movable through the opening 252″ of the discharge circuit board 250″ and includes an annular lip 261″ (FIG. 15) that prevents the plunger 260″ from falling through the opening. One end of a conductive switch 270″ is coupled to a top side of the second contact pad 257″ and the other end of the switch 270″ is movable relative to the first contact pad 256″ to electrically connect and disconnect the first contact pad 256″ and the second contact pad 257″. In particular, the switch 270″ extends from the second contact pad 257″, over the plunger 260″, and to the first contact pad 256″ such that upward movement of the plunger 260″ lifts off the switch 270″ from the first contact pad 256″, thereby breaking electrical contact between the switch 270″ and the first contact pad 256″. The switch 270″ is biased downward and removal of the lifting force of the plunger 260″ results in the switch 270″ moving downward to contact the first contact pad 256″, thereby creating electrical communication between the first contact pad 256″ and the second contact pad 257″ (e.g., through the switch 270″).

A retainer 210″ extending from a portion of the track 205″ maintains the position of the discharge circuit board 250″ in the first position prior to the battery assembly 200″ ever being coupled to any handle assembly 100″, that is, while the battery assembly 200″ is in a pre-installation state. The retainer 210″ also maintains the discharge circuit board 250″ in the second position after the battery assembly 200″ is removed from the handle assembly 100″, that is, while the battery assembly 200″ is in either of the post-installation state or the disposal state. In an aspect, the retainer 210″ includes a ramped surface extending into the track 205″ against which a proximal surface of the discharge circuit board 250″ rests to retain the discharge circuit board 250″ in the initial, first, position until a force is imparted upon the discharge circuit board 250″ to move the discharge circuit board 250″ along the track 205″ to the second position. When the discharge circuit board 250″ is moved to the second position, a backside of the retainer 210″ rests against a distal surface 254″ of the discharge circuit board 250″ to retain the discharge circuit board 250″ in the second position. The retainer 210″ retains the discharge circuit board 250″ in the second position while the battery assembly 200″ is coupled to the handle assembly 100″ (post-installation state) and after the battery assembly 200″ is removed from the handle assembly 100″ (disposal state). Although described as being positioned within the track 205″ and resting against a distal surface 254″ of the discharge circuit board 250″, the retainer 210″ may be configured in any manner suitable for retaining the discharge circuit board 250″ in the first and second positions.

The battery assembly 200″ also includes a first battery contact 220″ and a second battery contact 240″, a portion of each of which being positioned relative to the track 205″ in which the discharge circuit board 250″ is slidably disposed. In aspects, at least a portion of one of the first battery contact 220″ and the second battery contact 240″ is, or includes, a leaf spring. The discharge circuit board 250″ is movable relative to the first battery contact 220″ and the second battery contact 240″. With brief reference to FIG. 10, the first battery contact 220″ includes a first battery cell contact portion 226″, that is electrically coupled to a first terminal 206a″ of the battery cell 206″, and a first instrument contact portion 228″ to contact a first electrical terminal 128″ (FIG. 9) of the handle assembly 100″ when the battery assembly 200″ is coupled to the handle assembly 100″. Similarly, the second battery contact 240″ includes a second battery cell contact portion 246″, that is electrically coupled to a second terminal 206b″ of the battery cell 206″, and a second instrument contact portion 248″ to contact a second electrical terminal 148″ (FIG. 9) of the handle assembly 100″ when the battery assembly 200″ is coupled to the handle assembly 100″. The first electrical terminal 128″ and the second electrical terminal 148″ are electrically coupled to the electronic component 120″ of the handle assembly 100″ and are configured to close the electrical circuit, thereby providing electrical power to the electronic components (e.g., electronic component 120″) of the handle assembly 100″, when the battery assembly 200″ is coupled to the handle assembly 100″.

The handle assembly 100″ includes a handle finger 130″ extending from the handle housing 101″ and a protrusion 135″ extending from the handle finger 130″. A proximal portion of the handle finger 130″ includes a ramped surface 136″. The protrusion 135″ is positioned such that during the process of coupling the battery assembly 200″ to the handle assembly 100″, the protrusion 135″ engages the distal surface 254″ (FIG. 14) of the discharge circuit board 250″, moving the discharge circuit board 250″ along the track 205″ from the first position (FIG. 11) to the second position (FIG. 14) as the battery assembly 200″ is being connected to the handle assembly 100″. Additionally, as the battery assembly 200″ is being connected to the handle assembly 100″, a bottom portion of the plunger 260″ slides along the ramped surface 136″ of the handle finger 130″ and is forced upward, which in turn, causes the switch 270″ to disengage, and physically separate from, the first contact pad 256″ of the discharge circuit board 250″. In this configuration, power is supplied to the electronic component 120″ from the battery cell 206″ and the discharge circuit board 250″ is not discharging the battery cell 206″ because no connection is made between the first contact pad 256″ and the second contact pad 257″ due to the plunger 260″ lifting the switch 270″ and preventing the switch 270″ from contacting the first contact pad 256″.

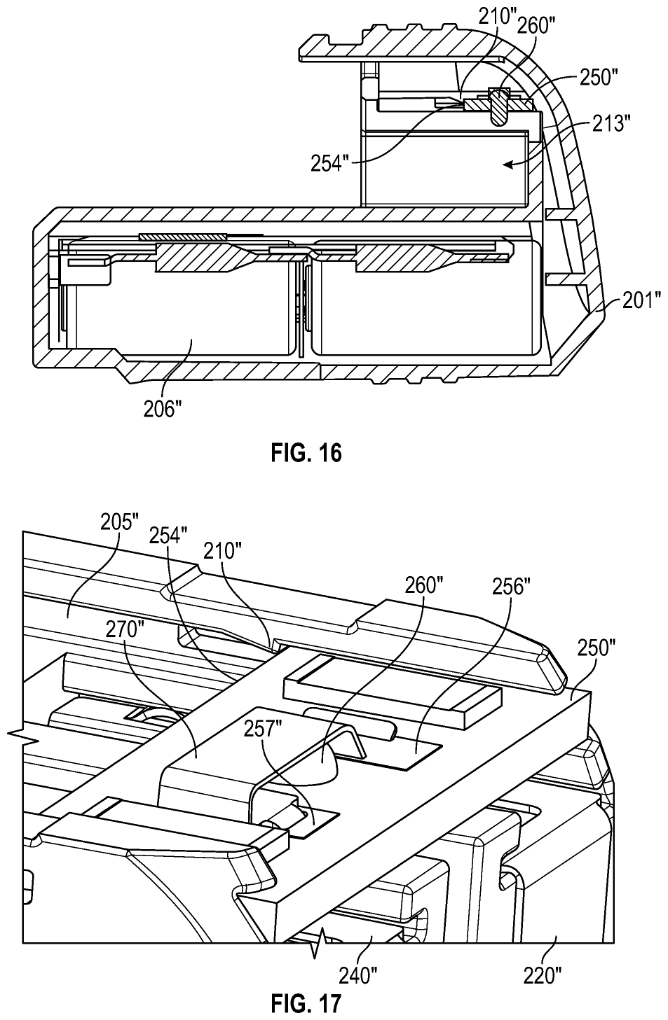

FIGS. 16-18 illustrate the battery assembly 200″ in a disposal state. In particular, as illustrated in FIGS. 16-18, subsequent to the battery assembly 200″ being removed from the handle assembly 100″, the discharge circuit board 250″ remains in the second position and is prevented from sliding to the first position by the abutment of the retainer 210″ against the distal surface 254″ of the discharge circuit board 250″. As the battery assembly 200″ is removed from the handle assembly 100″, the plunger 260″ slides down along the ramped surface of 136″ of the handle finger 130″, enabling the switch 270″ to move downward to contact the first contact pad 256″.

With the battery assembly 200″ removed from the handle assembly 100″, and the discharge circuit board 250″ in the second position, the switch 270″ is no longer prevented from creating a circuit between the first contact pad 256″ and the second contact pad 257″. Thus, in this configuration, with the second battery contact 240′ in conductive contact with a bottom side of the second contact pad 257″, the first battery contact 220″ in conductive contact with a bottom side of the first contact pad 256″, and the switch 270″ forming a connection between a top side of the first contact pad 256″ and a top side of the second contact pad 257″, the circuit between the battery cell 206″ and the resistive load of the discharge circuit board 250″ is closed. The resistive load of the discharge circuit board 250″ will discharge the battery cell 206″ until no more electrical energy is left in the battery cell 206″ of the battery assembly 200″.

The time to fully discharge the battery cell 206″ of the battery assembly 200″ depends on the capacity of the battery cell 206″ and the resistive load of the discharge circuit board 250″. In aspects, the resistive load of the discharge circuit board 250″ may be selectable, manually or automatically, to control the discharge rate. Resistors forming the resistive load of the discharge circuit board 250″ may be implemented in series and parallel to distribute electrical power as well as add safety redundancies to the discharging function of the discharge circuit board 250″.

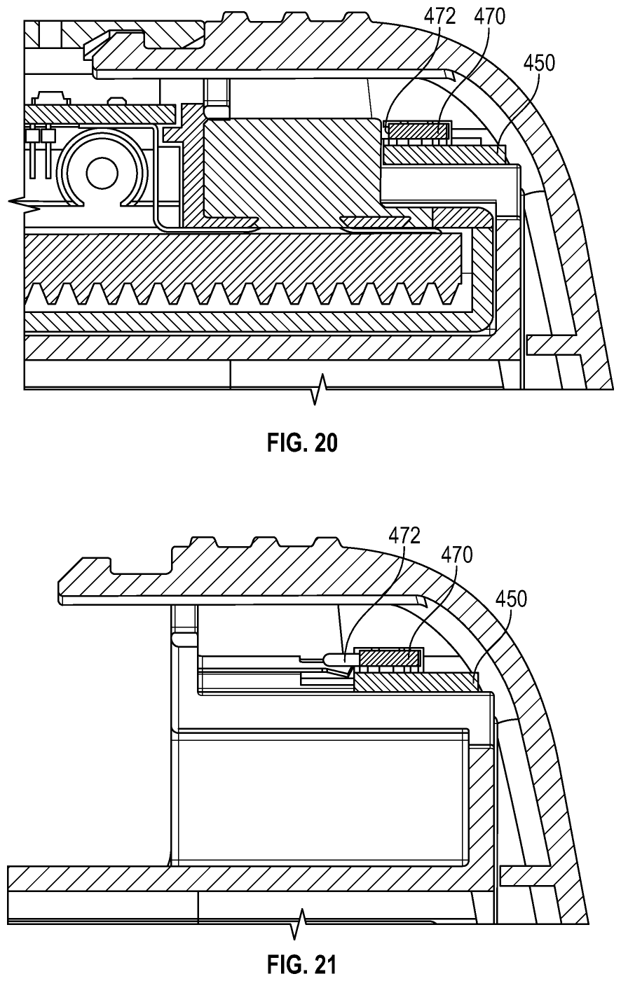

FIGS. 19-21 illustrate another embodiment of a discharge circuit board, shown as discharge circuit board 450, which may be implemented in battery assembly 200″ and which is similar to discharge circuit board 250″, with the exception that discharge circuit board 450 includes a detect switch 470 instead of switch 270″ to initiate the discharging of battery cell 206″. Therefore, only the differences between discharge circuit board 450 and discharge circuit board 250″ will be described for brevity.

Detect switch 470 includes a depressible button 472 which is biased outward and configured to be depressed inward when the battery assembly 200″ is coupled to the handle assembly 100″. When the depressible button 472 is in its biased outward position, the detect switch 470 is in an “ON” condition, and when the depressible button 472 is depressed the detect switch 470 is switched to an “OFF” condition. In the pre-installation state, the discharge circuit board 450 is in the first position and is not aligned with or contacting the battery contacts 490, and therefore, is not discharging the battery cell 206″ even though the detect switch 470 is in the “ON” condition. When the battery assembly 200″ is coupled to the handle assembly 100″, the discharge circuit board 450 is moved to the second position, where the discharge circuit board 450 is aligned with the battery contacts 490, and the detect switch 470 is switched from the “ON” condition to the “OFF” condition, thereby opening the circuit of the discharge circuit board 450 and disabling the discharge of the battery cell 206″.

Upon removal of the battery assembly 200″ from the handle assembly 100″, the discharge circuit board 450 is in the second position within the battery assembly 200″ and the depressible button 472 of the detect switch 470 is biased outwardly, placing the detect switch in the “ON” condition and initiating the discharge of the battery cell 206″.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A surgical instrument comprising:
a handle assembly including:
a handle housing;
an electronic component disposed within the handle housing, the electronic component including a first electrical terminal and a second electrical terminal; and
a handle finger extending from the handle housing; and
a battery assembly configured to removably couple to the handle assembly, the battery assembly including:
a battery housing;
a battery cell disposed within the battery housing;
a first battery contact and a second battery contact electrically coupled to the battery cell; and
a discharge circuit board configured to discharge the battery cell and movable within the battery housing between a first position and a second position, wherein the discharge circuit board is moved from the first position to the second position by the handle finger upon coupling the battery assembly to the handle assembly and wherein the discharge circuit board includes:
a first contact pad electrically coupled to the first battery contact;
a second contact pad electrically coupled to the second battery contact; and
a switch including a first end extending from the second contact pad and a second end movable relative to the first contact pad between a first condition where the second end is in conductive contact with the first contact pad and a second condition where the second end is not in conductive contact with the first contact pad.

2. The surgical instrument of claim 1, further comprising an elongated shaft extending from the handle assembly and an end effector operably coupled to the elongated shaft.

3. The surgical instrument of claim 1, wherein the battery assembly includes a retainer configured to maintain the discharge circuit board in the second position after the battery assembly is removed from the handle assembly.

4. The surgical instrument of claim 3, wherein the retainer is configured to maintain the discharge circuit board in the first position prior to the battery assembly being coupled to the handle assembly.

5. The surgical instrument of claim 1, wherein the discharge circuit board further includes a plunger positioned under the switch and movable through an opening defined through the discharge circuit board, the plunger configured to move the switch upward to disengage the switch from contact with the first contact pad.

6. The surgical instrument of claim 5, wherein the handle finger includes a ramped surface, and the plunger is configured to slide along the ramped surface as the battery assembly is being connected to the handle assembly.

7. The surgical instrument of claim 1, wherein the first contact pad is electrically coupled to the first battery contact and the second contact pad is electrically coupled to the second battery contact when the discharge circuit board is in the first position and the second position.

8. The surgical instrument of claim 1, wherein the discharge circuit board is configured to longitudinally slide along a track of the battery assembly.

9. The surgical instrument of claim 1, wherein the battery housing includes a battery connector, and the handle housing includes a handle connector configured to releasably mate with the battery connector.

10. The surgical instrument of claim 1, wherein the first battery contact is electrically coupled to a first terminal of the battery cell and the second battery contact is electrically coupled to a second terminal of the battery cell.

11. A battery assembly for use with a surgical instrument, the battery assembly comprising:
a battery housing;
a battery cell disposed within the battery housing;
a first battery contact and a second battery contact electrically coupled to the battery cell; and
a discharge circuit board movable within the battery housing between a first position in the battery housing and a second position in the battery housing, the discharge circuit board including:
a first contact pad electrically coupled to the first battery contact;
a second contact pad electrically coupled to the second battery contact; and
a switch including a first end extending from the second contact pad and a second end movable relative to the first contact pad between a first condition where the second end is in conductive contact with the first contact pad and a second condition where the second end is not in conductive contact with the first contact pad, wherein the discharge circuit board is configured to discharge the battery cell when the switch is in the second condition and the discharge circuit board is in the second position.

12. The battery assembly of claim 11, further comprising a retainer configured to maintain the discharge circuit board in the first position and the second position.

13. The battery assembly of claim 11, further comprising a track defined in the battery housing, wherein the discharge circuit board is configured to longitudinally slide along the track.

14. The battery assembly of claim 11, wherein the first battery contact is electrically coupled to a first terminal of the battery cell and the second battery contact is electrically coupled to a second terminal of the battery cell.

15. The battery assembly of claim 11, wherein the discharge circuit board further includes a plunger positioned under the switch and movable through an opening defined through the discharge circuit board, the plunger configured to move the switch upward to disengage the switch from contact with the first contact pad.

16. The battery assembly of claim 11, wherein the first contact pad is electrically coupled to the first battery contact and the second contact pad is electrically coupled to the second battery contact when the discharge circuit board is in the first position and the second position.

17. A surgical instrument comprising:
a handle assembly; and
a battery assembly configured to removably couple to the handle assembly, the battery assembly including:
a battery housing;
a battery cell disposed within the battery housing;
a first battery contact and a second battery contact electrically coupled to the battery cell; and
a discharge circuit board movable within the battery housing between a first position in the battery housing and a second position in the battery housing, the discharge circuit board including:
a first contact pad electrically coupled to the first battery contact;
a second contact pad electrically coupled to the second battery contact; and
a switch including a first end extending from the second contact pad and a second end movable relative to the first contact pad between a first condition where the second end is in conductive contact with the first contact pad and a second condition where the second end is not in conductive contact with the first contact pad, wherein the discharge circuit board is configured to discharge the battery cell when the switch is in the second condition and the discharge circuit board is in the second position.

18. The surgical instrument of claim 17, wherein the battery assembly includes a retainer configured to maintain the discharge circuit board in the first position prior to the battery assembly being coupled to the handle assembly and maintain the discharge circuit board in the second position after the battery assembly is removed from the handle assembly.
